(19) United States
(12) Patent Application Publication
(10) Pub. No.: US 2003/0008005 A1
(43) Pub. Date: Jan. 9, 2003

(54) Sublingual administration of dihydroergotamine for the treatment of migraine

(75) Inventor: Neal R. Cutler, Los Angeles, CA (US)

Correspondence Address: MEDLEN & CARROLL, LLP, 101 HOWARD STREET, SUITE 350, SAN FRANCISCO, CA, 94105, US

(73) Assignee: R.T. Alamo Ventures, Inc.

(21) Appl. No.: 09/899,412

(22) Filed: Jul. 5, 2001

(51) Int. Cl. A61K 9/46; A61K 31/48

(52) U.S. Cl. 424/466; 514/288

(57) ABSTRACT

The present invention is an improvement in the treatment of migraine headaches. By administering dihydroergotamine sublingually, major limitations of past treatments are circumvented thereby allowing for higher efficacy and fewer side effects of treatment at lower doses.

### FIELD OF THE INVENTION

The present invention relates to the use of dihydroergotamine for the treatment of migraine headaches via sublingual administration.

### BACKGROUND OF INVENTION

Migraine is the most common neurological condition in the developed world. It affects 10% of the U.S. population and is more prevalent than diabetes, epilepsy and asthma combined. Migraine is more than just a headache. It can be a debilitating condition which has a considerable impact on the quality of life of sufferers and their families. Attacks can be completely disabling, forcing the sufferer to abandon everyday activities for up to 3 days. Even in symptom-free periods, sufferers may live in fear of the next attack. Migraine attacks normally last between 4 and 72 hours and sufferers are usually symptom free between attacks.

Migraine is believed to be caused by the release of a chemical called serotonin or 5HT into the bloodstream from its storage sites in the body, resulting in changes in the neurotransmitters and blood vessels in the brain. This causes the pain neurons in the blood vessel wall to become irritated. Exactly what cause the release of serotonin is still a subject for research and debate. However, certain factors have been identified which can trigger attacks in susceptible people. Some of these are stress or sometimes the relief of stress, lack of food or infrequent meals, foods containing monosodium glutamate, caffeine and tyramine, certain specific foods like chocolate, citrus fruits or cheese, alcohol (especially red wine), overtiredness (physical or mental), changes in sleep patterns (e.g., late nights or a weekend lie-in), or hormonal factors (e.g., monthly periods, the contraceptive pill or hormonal changes in males and females as they age), etc.

Migraine triggers are numerous and varied and occur in combinations peculiar to each individual. It should be noted that not all migraineurs will be affected by all of the above triggers and possibly by none of them. Everyone has the capacity to suffer from migraine but in some people, most probably because of a genetic predisposition, the threshold at which attacks occur is lower. Migraineurs come from all walks of life, all areas of the world and ethnic groups, and all social classes.

Migraine is a complex condition and a treatment which is successful for one patient may have no effect on another. It is therefore important to continue to develop new methods of treatment and new modes of administration of compounds that show therapeutic potential in mitigating migraines.

What is needed are compounds and drugs that are effective for the treatment of migraines in a formulation that allows for better drug delivery and ease of use by the patient.

### SUMMARY OF INVENTION

The present invention relates to the use of dihydroergotamine via sublingual administration for the treatment of migraine headaches. The present invention contemplates both prophylactic and acute treatment of migraine.

Current methods of administering DHE to migraine sufferers have major efficacy limitations. For example, due to degradation in the gastrointestinal track and low adsorption of the drug, oral forms of DHE have to be administered in large doses of about 20-30 mg. Such high dosing causes nausea, vomiting and other side effects in many patients. Much of the DHE is subject to pre-systemic and first pass metabolism. Because of this, is it estimated that as little as 2% of the active drug actually reaches the blood stream. Likewise, intranasal administration of DHE is hampered with significant limitations. A 4 mg intranasal dose of a pharmaceutical salt of DHE is hampered by a large administered volume and subsequent low systemic absorption of the drug and by oral ingestion of large quantities of the dose.

Injectable forms of DHE are also available for the treatment of migraines. Although the direct administration of DHE into the blood stream allows for a lower dosage as compared to other methods of administration, the inconvenience of an office visit for an injection or problems with the self-administration of injectables are self evident.

Although not limited to any particular mechanism, the present invention contemplates the sublingual administration of DHE in a drug delivery system that adjusts the pH of the local environment thereby allowing for the ready absorption of DHE into the blood stream. This is because the adjustment of the pH permits the conversion of DHE to the more permeable base form. Additionally, the quick-dissolve nature of the formulation of the present invention also aids in the rapid absorption of DHE into the blood stream.

The following description does not limited the present invention to any particular mechanism and is only for illustrative purposes. 5HT agonists (sometimes known as triptans) act directly to correct the serotonin imbalance in the brain during a migraine attack. Dihydroergotamine (DHE), however, belongs to the group of medicines known as ergot alkaloids. DHE is involved in vasoconstriction (narrowing of arteries and veins that supply blood to the head). Dihydroergotamine is also involved in altering blood flow patterns that are associated with vascular headaches.

Migraine drugs are often not suitable for many patients for a variety of reasons. One of the common reasons is that the drugs are given in incompatible or ineffective modes of administration. Often times the mode of administration may limit the effectiveness of the drug. Furthermore, some patients may have difficulty in taking these drugs due to the limited formulations in which they are made available.

Current modes of administration of DHE for migraine necessitate large doses of DHE to be used, e.g., 20-30 mg for oral administration and 4 mg for nasal administration, respectively. Large doses may cause adverse side effects in the patient. For example, nausea and vomiting are a common side effects. One way to reduce the severity of side effects would be to lower the dose of DHE administered to the patient while still maintaining a therapeutic level of DHE at the target site. A sublingual formulation of DHE in the base form of the drug would permit the use of lower doses of DHE since a greater portion of the medication would be absorbed directly into the blood stream thereby allowing a direct route to the afflicted target area. For example, as compared to the current marketed nasal spray formulation, the present invention contemplates about a 25% reduction in dose, giving a preferred dose in the range of about 2.5 to 3.5 mg. In another embodiment, as compared to the current marketed nasal spray formulation, the present invention contemplates about a 50% reduction in dose, giving a preferred dose in the range of about 1.5 to 2.5 mg. In yet another embodiment, the present invention contemplates about a 75% in dose as compared to the current marketed nasal spray formulation, giving a preferred dose range of about 0.75 to 1.5 mg. In still yet another embodiment, the present invention contemplates about a 90% reduction in dose over the current marketed nasal spray formulation, giving a dose range of about 0.2 to 0.75 mg. Sublingual formulations of DHE will also allow for ease of administration by the patient. Of course, it will be clear to one practiced in the art that the dosages of DHE administered by the methods contemplated by the present invention may need to be adjusted depending on the weight, age and physical condition of the patient and use of other medications by the patient, etc.

The present invention comprises treating a patient suffering from a migraine headache with a therapeutic dose of dihydroergotamine, or a pharmacologically acceptable salt thereof, in a sublingual formulation. It is contemplated that the DHE may be in the form of dihydroergotamine mesylate or any pharmaceutically acceptable salt. It is contemplated that the sublingual administration of DHE may be made with a fast dissolve formulation.

Although the present invention is not limited to any particular mechanism, it is believed that the adjustment of the pH of the environment of the sublingual area will convert the administered DHE to the readily absorbable DHE base. The pH of DHE in solution is typically in the range of 3.2-4.0. It is contemplated that the fast dissolve formulation comprise at least one component the will adjust the pH of the local environment of the sublingual area. The preferred pH of the sublingual environment for administration of DHE is above 4.2. The more preferred pH of the sublingual environment for the administration of DHE is between 5 and 7.

Sublingual administration of a fast dissolve DHE may take many forms. It one embodiment it is contemplated that DHE is in the form of a tablet or packed powder. The sublingual administration of DHE may comprise a medical device such as a patch. The patch may be placed under the tongue. The patch may have adhesive qualities to prevent the movement, loss or swallowing of the patch. The patch may be ingestible in case of accidental swallowing or to allow for easy disposal of the patch. In another embodiment the patch may be removed from under the tongue after the prescribed time. In yet another embodiment, the sublingual administration of DHE may take the form of a paste or gel. The paste or gel would be applied under the tongue. The viscosity of the paste or gel can be adjusted to allow for the retention under the tongue. In another embodiment, it is contemplated that the present invention is a liquid. It is further contemplated that the liquid is in the form of a spray or drops.

In a particularly preferred formulation in accordance with the present invention there is provided a hard, compressed, rapidly dissolving tablet adapted for direct sublingual dosing. The tablet includes particles made of an active ingredient and a protective material. These particles are provided in an amount of between about 0.01 and about 75% by weight based on the weight of the tablet. The tablet may also include a matrix made from a nondirect compression filler, a wicking agent, and a hydrophobic lubricant. The preferred tablet matrix comprises at least about 60% rapidly water-soluble ingredients based on the total weight of the matrix material. The preferred tablet has a hardness of between about 15 and about 50 Newtons, a friability of less than 2% when measured by U.S.P. and is adapted to dissolve spontaneously in the mouth of a patient in less than about 120 seconds and thereby liberate said particles and be capable of being stored in bulk.

In yet another preferred formulation the compressed rapidly dissolving tablet comprises effervescent agents. These effervescent agents allow enhanced adsorption of the active ingredient across the mucosal membranes in the sublingual cavity. An example of effervescent pharmaceutical compositions suitable for use in conjunction with the present invention are the compositions described in Pather, U.S. Pat. No. 6,200,604, which is incorporated herein by reference.

In one embodiment of the present invention, it is contemplated that DHE is combined with inactive ingredients. Such ingredients may be necessary, e.g., to add bulk to the pharmaceutical preparation, to bind the preparation, to add color or flavor to the preparation, to prevent degradation or growth of contaminants, etc.

It is also contemplated that the present invention comprise other active ingredients in addition to DHE which may be added to the pharmaceutical preparation of the present invention. The addition of any other active ingredient or ingredients is contemplated except where limited by the prior art. Such added active ingredients may augment the effectiveness of DHE in alleviating or ameliorating migraines. For example, it is contemplated that analgesics or anesthetics may be added to the pharmaceutical preparation. In a particular embodiment, non-steroidal antiinflammatory drugs (NSAID) are contemplate as additional active ingredients. The present invention is not limited to any particular type of NSAID. In another embodiment, the present invention contemplates the addition of active ingredients that may help to alleviate any side effects of the medication or of the migraine. In one embodiment the added agent may alleviate nausea and vomiting. It is contemplated that the other active ingredients be administered in combination with the sublingual dose of DHE. Such co-administration may be sublingual, oral, rectal, buccal, injectable, nasal, transcutaneous, etc.

Definitions

“Migraine” and “migraine headache” is defined herein as a recurrent, throbbing headache generally, but not always, felt on one side of the head.

“Sublingual” is defined herein as beneath or concerning the area under the tongue.

“Sublingual administration” is defined herein as the therapeutic administration of a pharmaceutical composition under the tongue. Such pharmaceutical compositions may be formulated so that the dissolve when placed under the tongue. The pharmaceutical compositions may dissolve slowly, moderately quickly or rapidly. Additionally, such sublingual formulations may constitute a medical device that comprises the therapeutic compound and is removed from under the tongue and taken out of the mouth once the compound has dissolved or after a prescribed amount of time.

“Dihydroergotamine,” “DHE,” “dihydroergotamine mesylate” and synonyms thereof, shall be defined as a therapeutic amount of dihydroergotamine or a pharmaceutical acceptable derivative or salt thereof.

“Migraine-ameliorating effective amount” shall be defined as an amount of dihydroergotamine which effects a prophylactic or therapeutic response in the patient in need of such a response over a reasonable time frame (e.g., between 1 and 4 hours), causing either a diminution or an eradication of one or more of the symptoms of migraine (e.g., a reduction in throbbing or pain).

“Analgesic” shall be defined as a chemical substance capable of causing diminished sensitivity to pain.

“Vasoconstrictor” shall be defined as a chemical substance that induces the narrowing of the lumen of blood vessels, i.e., vasoconstriction. “Non-vasodilating” shall be defined as a compound, drug, pharmaceutical, treatment or therapy that does not induce vasoconstriction.

“Therapeutic formulation” shall be described as a pharmaceutical composition comprising at least one active ingredient along with other optional ingredients useful in, for example, binding, flavoring, coloring, preserving, stabilizing, increasing self life, adding structural rigidity, adding desired mouth feel, adding desired mouth consistency, aiding in regulating dissolve rate, adjusting the pH of the local environment or adding adhesive qualities to help prevent movement of the therapeutic formulation in the mouth.

“Water-soluble” in accordance with the present invention has its usual meaning. However, “rapidly water-soluble” shall be defined as the ingredient in question will dissolve in a time frame as defined below under “fast dissolve”. For example, a very fine grained or powdered sugar known as a nondirect compression sugar may be used as a filler in the matrix of the present invention. This material, in part because of its chemical composition and in part because of its fine particle size, will dissolve readily in the mouth in a mater of seconds once it is wetted by saliva.

“Dosage form,” in accordance with the present invention, includes tablets and “slugged cores” used in capsules or caplets (a hybrid tablet/capsule).

“Dissolvable,” shall be defined as describing the action of the dosage form as it is held in the mouth. In this case, the dosage form gets continuously smaller in a process which is conceptually analogous to melting. While the dosage form may also disintegrate into smaller pieces to some extent, particularly where a relatively greater amount of a wicking agent or effervescent disintegrant is used, that is not its principal mechanism.

“Rapidly dissolve(able)”, “rapid(ly) dissolving” and “fast dissolve(able)” shall be defined as the rapidly water-soluble ingredients will dissolve sufficiently to allow at least 50% solubilization of the active ingredient or ingredients in 180 seconds or less, preferably 90 seconds or less and most preferably 60 seconds or less.

“Effervescent agent” shall be defined as refers to compounds that evolve gas. The preferred effervescent agents evolve gas by means of a chemical reaction which takes place upon exposure of the effervescent agent to an aqueous solution such as water or saliva.

“Administered in combination”, “co-administered” or equivalent terms, shall be defined as pharmaceuticals that are administered simultaneously or sequentially with DHE. The pharmaceuticals administered need not be in the same dosage form (i.e., sublingual) as the DHE. “In combination with” DHE shall be defined as the administration of the other drug either simultaneously or sequentially with DHE.

“pH adjusting agent” shall be defined as a compound that, alters or adjusts the pH of the local environment. In the context of the present invention, a “pH adjusting agent” alters or adjusts the pH of the sublingual area upon dissolving. The pH of DHE in solution is typically in the range of 3.2-4.0. It is contemplated that the fast dissolve formulation of the present invention comprise at least one component the will adjust the pH of the local environment of the sublingual area. The preferred pH of the sublingual environment for administration of DHE is above 4.2. The more preferred pH of the sublingual environment for the administration of DHE is between 5 and 7.

“Reduced” and “reduced symptoms” shall be defined as a lessening of symptoms to a noticeable degree by either the subject or medical professional. In the context of the present invention reduced symptoms shall mean, for example, the lessened severity of the subject's migraine headache. “Lessened severity” shall be defined, for example, as reduced pain, reduced throbbing, an increased ability for the subject to perform his or her normal routine, etc. It is not necessary, in the context of the present invention, for the treatment to relieve all symptoms of the migraine or to completely relieve the symptoms of the migraine.

### GENERAL DESCRIPTION OF INVENTION

The present invention is an improvement in the treatment of migraine headaches. Although the present invention is not limited to any particular mechanism, by administering dihydroergotamine in a lower dose sublingually, major limitations of past treatments are circumvented thereby allowing for higher efficacy and fewer side effects.

Although the present invention is not limited to any particular mechanism, migraines are believed to be caused by a rapid widening and narrowing of blood vessel walls in the brain and head. This causes the pain neurons in the blood vessel wall to become irritated. Blood vessels in the scalp are often involved. The following items and events (precipitant) have been reported to cause migraine attacks: hunger, cheese, changes in weather, nuts, fatigue, avocados, oral contraceptives, chocolate, menstrual periods, food cured with nitrates (e.g., hot dogs), emotional stress, meat tenderizers (e.g., MSG), and alcoholic beverages. It is not known why some individuals have migraines and others do not.

Migraines are classified as either a classical migraine or a common migraine. See, e.g., “Remington's Pharmaceutical Sciences”, 17th ed., Mack Publishing Company (1985), p. 946 and Goodman and Gilman's “The Pharmaceutical Basis of Therapeutics”, 8th ed., McGraw-Hill, Inc. (1990), pp. 944-947. A common migraine is much more likely to occur than a classical migraine. The classical migraine is associated with objective prodromal neurological signs and symptoms involving a headache that is preceded by a slowly expanding area of blindness surrounded by a sparkling edge that increases to involve up to one half of the field of vision of each eye. When the blindness clears up after approximately 20 minutes, it is often followed by a severe one-sided headache with nausea, vomiting and sensitivity to light. The common migraine is an attack without prodromal symptoms and begins as a slowly developing pain in the form of a headache that transforms into a mounting throbbing pain made worse by the slightest movement or noise. The pain is often on one side of the head only and usually occurs with nausea and sometimes vomiting.

The length of migraine is from about four hours to three days. Examples of causes of migraine are: stress related (e.g., anxiety, anger, worry, excitement, shock, depression), overexertion, changes of routine and changes of climate, food-related (e.g., chocolate, cheese and other dairy products, red wine, fried food and citrus fruits), sensory-related (e.g., bright lights or glare, loud noises and intense or penetrating smells), menstruation, contraceptive drugs and male or female age related hormonal changes.

Antimigraine drugs are well-known. See, e.g., U.S. Pat. Nos. 4,650,810, 4,914,125, 4,916,125, 4,994,483, 5,021,428, 5,200,413, 5,242,949, 5,248,684, 5,273,759, 5,317,103, 5,364,863, 5,399,574, 5,434,154, 5,441,969, 5,464,864, 5,466,699, 5,468,768, 5,491,148 and 5,494,910. Antimigraine drugs most commonly used in treatment of migraine fall into the following groups: beta-blocking agents, calcium channel blocking agents, antidepressants, selective 5-HT₁ agonists (sumatriptan), sedatives, local anesthetics, adrenergic blocking agents and mixtures of these.

The success of triptans in the treatment of migraine is limited. Such drugs (e.g., rizatriptan) show only a 60-70% efficacy.

Some antimigraine drugs may have direct, or indirect effect on the 5-hydroxytryptamine (5-HT) receptor system. The 5-HT receptor system is a potent intracranial vasoconstrictor. 5-HT receptors are presently delineated into three major subclassifications—5-HT₁, 5-HT₂, and 5-HT₃—each of which may also be heterogeneous. The receptor mediates vasoconstriction in the carotid vascular bed and thereby modifies blood flow therein. The various classes of compounds have been proposed as 5-HT agonists or antagonists for therapeutic use of migraine, but these have not always been specific to a particular type of 5-HT receptor.

Management of migraine is complicated by the lack of a single therapy which is effective in all patients with the same migraine type and by the need to select either an abortive or prophylactic method of treatment for these migraines. Further complications involves the current use of drugs that cause dependence with extended use. Another important consideration is that the more effective antimigraine agents in current use produce severe use-limiting side effects with long term usage.

Thus, there is a need for a safe and effective drug for the treatment of migraine and related disorders which can be used either prophylactically or to alleviate an established migraine that minimizes side effects preferably by allowing for the use of lower doses of the therapeutic compound.

### DETAILED DESCRIPTION OF PREFERRED EMBODIMENTS

The present invention is directed, among other things, to methods of treating migraine by the sublingual administration of a migraine-ameliorating effective amount of an dihydroergotamine or effective pharmaceutical salt thereof. While the precise mechanism by which the sublingual administration of a migraine-ameliorating effective amount of dihydroergotamine relieves migraine is unknown and without limiting the invention to any particular theory, it is believed that the treatment is effective because of its vasoconstrictive properties.

Sublingual administration is the preferred method of administration of the present invention. Although the present invention is not limited to any particular mechanism, is believed that this method of administration allows for efficient transfer of the drug into the blood stream thereby maximizing the degree to which dihydroergotamine is absorbed for therapeutic intervention and minimizing the degree to which dihydroergotamine is absorbed orally. In other words, an advantage of such sublingual administration and the absorption of DHE through the sublingual mucosa is the effectiveness of lower doses of dihydroergotamine than intranasal dosing thereby, reducing any adverse effect. A further advantage of sublingual administration is a low absorption through a systemic route thereby reducing systemic side effects.

Several pharmaceutically acceptable methods of sublingual administration are well-known to those who are skilled in the art. The choice of method of sublingual administration method will be determined in part by the patient. The following methods of administration are merely exemplary and in no way limit the present invention.

Liquids can be used for the sublingual administration of DHE. Liquid formulations suitable for sublingual administration can consist of (a) solutions, such as a migraine-ameliorating effective amount of the agent dissolved in diluents such as water, or saline; (b) suspensions in an appropriate liquid; (c) suitable emulsions, all of which can be administered in suitable ways, including drops and sprays. These formulations may also contain excipients as are known in the art. Formulations can also include gels, ointments and the like, containing, in addition to the active ingredient, such excipients as are known in the art. All of these formulations can be administered in suitable ways, including by spraying, dripping, painting or squirting under the tongue.

DHE, alone or in combination with other suitable components, can also be made into aerosol formulations to be administered via a spray. These aerosol formulations can be placed into pressurized acceptable propellants, such as dichlorodifluoromethane, propane, nitrogen, and the like. They may also be formulated as pharmaceuticals for non-pressured preparations such as in a nebulizer or an atomizer.

In a preferred embodiment, DHE is administered sublingually in liquid form, most preferably in a flavored or unflavored physiological saline solution. In a preferred embodiment, the solution is administered as drops. In another preferred embodiment, DHE in liquid form is administered as a spray under the tongue.

In a preferred embodiment, DHE is administered as a solution comprising about 0.01% to about 0.5% DHE. More preferably, the solution is a physiological saline solution. Preferably, the amount of solution administered is about 0.01 ml to about 1 ml. More preferably, the amount of solution is about 0.25-0.5 ml.

In another preferred embodiment, the sublingual formulation of DHE comprises a hard or compressed powder tablet designed to dissolve under the tongue in about 30 to 120 seconds as disclosed in U.S. Pat. No. 6,221,392 to Khankari, et al., incorporated herein by reference. In another embodiment, the formulation of the hard tablet has a low grit component for an organoleptically pleasant mouth feel. The active component of the tablet is contained within a protective material. The particles are then added to a matrix comprising rapid dissolving, water soluble ingredients. In this regard, the present invention relates to a hard, compressed, rapidly dissolvable dosage form adapted for direct sublingual dosing. The dosage form includes an active ingredient and a matrix. The matrix is composed of at least a nondirect compression filler and a lubricant. The dosage form is adapted to rapidly dissolve under the tongue of a patient and thereby liberate the active ingredient. Preferably, the dosage form has a friability of about 2% or less when tested according to the U.S.P. The dosage form also preferably has a hardness of 15-50 Newtons (N).

The dosage forms described above are able to dissolve rapidly under the tongue of the patient, with a minimum of grit or other organoleptically unpleasant species. Moreover, because the dosage forms are hard and have low friability they can be handled and packaged like other, nonrapidly dissolving dosage forms.

Therefore, in another aspect of the present invention, there is provided a method of making a packaged, sublingually dissolvable dosage form. The method includes the steps of: (a) forming a mixture including an active ingredient (e.g., DHE) and a matrix including a nondirect compression filler and a lubricant; (b) compressing the mixture to form a plurality of hard, compressed, rapidly disintegrable dosage forms including the active ingredient distributed in the sublingually dissolvable matrix; and (c) storing the dosage forms in bulk prior to packaging. In a preferred particularly preferred embodiment, the dosage forms are then packaged in a lumen of a package such that there more than one per package. Direct compression is the preferred method of forming the dosage forms. There is also provided hereby an openable and recloseable package containing a plurality of hard, compressed, rapidly dissolving tablets adapted for direct oral dosing as described above.

The protective materials used in accordance with the present invention may include any of the polymers conventionally utilized in the formation of microparticles, matrix-type microparticles and microcapsules. Among these are cellulosic materials such as naturally occurring cellulose and synthetic cellulose derivatives; acrylic polymers and vinyl polymers. Other simple polymers include proteinaceous materials such as gelatin, polypeptides and natural and synthetic shellacs and waxes. Protective polymers may also include ethylcellulose, methylcellulose, carboxymethyl cellulose and acrylic resin material sold under the registered trademark EUDRAGIT by Rhone Pharma GmbH of Weiterstadt, Germany.

In another preferred embodiment, the present invention comprises an effervescent agent. The effervescent agent is provided in an amount of between about 5% and about 95% by weight, based on the weight of the finished tablet, and more preferably in an amount of between about 30% and about 80% by weight. It is particularly preferred that sufficient effervescent material be provided such that the evolved gas is more than about 5 cm³ but less that about 30 cm³, upon exposure of the tablet to an aqueous environment. Sublingual compositions comprising effervescent agents are provided in Pather U.S. Pat. No. 6,200,604 which is incorporated herein by reference.

In one embodiment, the effervescent agent(s) of the present invention evolve carbon dioxide. Although not limited to a particular mechanism, this reaction is most often the result of the reaction of a soluble acid source and a source of carbon dioxide such as an alkaline carbonate or bicarbonate. The effervescent agent(s) of the present invention is not always based upon a reaction which forms carbon dioxide. The acid sources may be any which are safe for human consumption and may generally include food acids, acid and hydrite antacids such as, for example: citric acid, tartaric, amalic, fumeric, adipic and succinics. Carbonate sources include dry solid carbonate and bicarbonate salt such as, preferably, sodium bicarbonate, sodium carbonate , potassium bicarbonate and potassium carbonate, magnesium carbonate and the like. Reactants which evolve oxygen or other gasses which are safe for human consumption are also considered within the scope of the present invention.

The dosage forms of the present invention may also include a pH adjusting substance. Although the present invention is not limited to any particular mechanism, the pH of aqueous environments can influence the relative concentration of the ionized and unionized forms of the drug present in solution. The pH of the local environment, e.g., saliva in immediate contact with the tablet and any drug that may have dissolved from it, may be adjusted by incorporating in the tablet pH adjusting substances which permit the relative portions of the ionized and unionized forms of the drug to be controlled. In the present invention it is contemplated that the pH of the micro-environment will be altered such that the DHE mesylate salt will exist as the DHE base.

Suitable pH adjusting agents for use in the present invention include, but are not limited to, any weak acid or weak base in amounts additional to that required for the effervescence or, preferably, any buffer system that is not harmful to the oral mucosa. Suitable pH adjusting substance for use in the present invention include, but are not limited to, any of the acids or bases previously mentioned as effervescent compounds, disodium hydrogen phosphate, sodium dihydrogen phosphate and the equivalent potassium salt.

The dose administered in the context of the present invention should be a migraine-ameliorating effective amount of DHE. Current modes of administration of DHE for migraine (e.g., oral and nasal administration) necessitate large doses of DHE to be used. Large doses may cause adverse side effects in the patient. Nausea and vomiting are a common side effects. More severe side effects include stoke, heart palpatations and, rarely, death. One way to reduce the severity of side effects would be to lower the dose of DHE administered to the patient while still maintaining a therapeutic level of DHE at the target site. A sublingual formulation of DHE would permit the use of lower doses of DHE since a greater portion of the medication would be absorbed directly into the blood stream thereby allowing a direct route to the afflicted target area. For example, as compared to the current marketed nasal spray formulation, the present invention contemplates about a 25% reduction in dose, giving a preferred dose in the range of about 2.5 to 3.5 mg. In a more preferred embodiment, as compared to the current marketed nasal spray formulation, the present invention contemplates about a 50% reduction in dose, giving a preferred dose in the range of about 1.5 to 2.5 mg. In yet a more preferred embodiment, the present invention contemplates about a 75% in dose as compared to the current marketed nasal spray formulation, giving a preferred dose range of about 0.75 to 1.5 mg. In yet another preferred embodiment, the present invention contemplates about a 90% reduction in dose over the current marketed nasal spray formulation, giving a dose range of about 0.2 to 0.75 mg. Sublingual formulations of DHE will also allow for ease of administration by the patient.

Preferably, the symptoms of the migraine are relieved within about 5 to about 120 minutes after administration of a sublingual dose of DHE, and more typically within about 10 to about 30 minutes, and if they are not relieved within about 120 minutes, a second dose can be administered.

The methods of the invention further include a method of treatment of migraine comprising the sublingual administration of DHE, in combination with the administration of at least one antiinflammatory compound. Antiinflammatory compounds include steroids, particularly glucocorticoids, for example, cortisol, cortisone, prednisolone, dexamethasone and the like; and nonsteroids, particularly salicylates (such as aspirin), pyrazolon derivatives (such as phenylbutazone), indomethacin and sulindac, fenamates, and propionic acid derivatives (such as ibuprofen). In a preferred embodiment, the nonsteroidal antiinflammatory agent ketorolac tromethamine in a 0.5% solution or diclofenac sodium in a 0.1% solution is administered.

The methods of the invention further include a method of treatment of migraine comprising the sublingual administration of DHE in combination with the administration of at least one non-DHE antimigraine drug, such as pizotifen, propranolol, valproate, amitriptyline, methylsergide, sumatriptan or other triptans and flunarizine.

The present invention is not limited in the method in which DHE is administered in combination with other pharmacological agents. For example, the other pharmacological agents may be administered concurrently or sequentially with DHE. Likewise, the other pharmacological agents may be administered by modes of administration other that sublingually. For example, they may be administered orally, buccally, intravenously, subcutaneously, nasally or via the rectum. Additionally, they may be administered in fast release, slow release or controlled release formulations.

The following examples further illustrates the present invention but, of course, should not be construed as in any way limiting its scope.

### EXAMPLES

### Example 1

This example illustrates a method of administering a migraine-ameliorating amount of DHE sublingually.

A dose of a DHE sublingual compound is placed by the patient or medical professional under the tongue. The compound is allowed to dissolve fully. If the symptoms are not relieved within 30 to 60 minutes, a second dose is administered.

### Example 2

This example illustrates a method of administering a migraine-ameliorating amount of DHE sublingually.

This experiment utilizes two test groups of patients. All of the patients are suffering from migraines. One group receives a dose of sublingual DHE, the other group receives a placebo. A dose of a DHE sublingual compound is placed by the patients or medical professional under the tongue. The compound is allowed to dissolve fully. If the symptoms are not relieved within 20 to 40 minutes, a second dose is administered. Results are compared between the test group and the placebo group. The results show greater amelioration of migraine symptoms in the test group as compared to the control group. The difference in amelioration of migraine symptoms are statistically significant. Additionally, the results show the effectiveness of DHE in treating migraine as compared to a placebo.

### Example 3

This example compares the effect on migraine of subcutaneous injection vs. the sublingual administration of the present invention.

This experiment utilizes three test groups of patients. All of the patients are suffering from migraines. One group receives a 1 mg dose of sublingual DHE, the second group receives a 1 mg subcutaneous injection of a pharmaceutical formulation comprising DHE and the third group receives a placebo. A dose of a DHE sublingual compound is placed by the patient or medical professional under the tongue, injected subcutaneously or the placebo is given. The sublingual compound is allowed to dissolve fully. Results are compared between the sublingual test group and the subcutaneous test group. The results show equivalent amelioration of migraine symptoms between the two groups indicating that the easier sublingual administration of the present invention is as effective as the more difficult subcutaneous administration of DHE. Additionally, the results show the effectiveness of DHE in treating migraine as compared to the placebo.

### Example 4

This example compares the effect on migraine of nasal administration of DHE vs. the sublingual administration of the present invention.

This experiment utilizes three test groups of patients. All of the patients are suffering from migraines. One group receives a 1 mg dose of sublingual DHE, the second group receives a 4 mg nasally administered dose of a pharmaceutical formulation comprising DHE and the third group receives a placebo. A dose of a DHE sublingual compound is placed by the patient or medical professional under the tongue, given nasally or a placebo is given. The sublingual compound is allowed to dissolve fully. Results are compared between the sublingual test group and the nasal test group. The results show equivalent amelioration of migraine symptoms between the two groups indicating that the lower dose sublingual administration of the present invention is as effective as the higher dose nasal administration of DHE. Additionally, the results show the effectiveness of DHE in treating migraine as compared to the placebo.

As is evident from the foregoing, the present invention contemplates novel treatment methods for migraines comprising the sublingual administration of dihydroergotamine. These novel methods allow for the circumvention of major limitations of past treatments thereby allowing for higher efficacy and fewer side effects of treatment at lower doses.

1. A method of treating migraines, comprising:
a) providing i) a patient having one or more symptoms of a migraine and ii) a formulation comprising dihydroergotamine;
b) administering said formulation to said patient sublingually under conditions such that said one or more symptoms of said migraine are reduced.

2. The method of claim 1, wherein said dihydroergotamine is a pharmaceutically accepted salt.

3. The method of claim 1, wherein said dihydroergotamine is a pharmaceutically accepted base.

4. The method of claim 1, wherein said sublingual administration is via a liquid.

5. The method of claim 4, wherein said liquid is administered by a spray.

6. The method of claim 4, wherein said liquid is administered by drop.

7. The method of claim 1, wherein said sublingual administration is via a paste or gel.

8. The method of claim 1, wherein said sublingual administration is via a tablet or compressed powder.

9. The method of claim 1, wherein the method additionally comprises the coadministration of at least one other pharmaceutically accepted compound.

10. The method of claim 9, wherein the pharmaceutically accepted compound is a non-vasodilating treatment for migraine.

11. The method of claim 1, wherein said therapeutic formulation of DHE additionally comprises a pain reliever.

12. The method of claim 1, wherein the sublingual administration is via a fast dissolve formulation.

13. The method of claim 1, wherein said formulation additionally comprises at least one effervescent agent.

14. The method of claim 1, wherein said formulation additionally comprises at least one pH adjusting agent.
